(19) United States
(12) Patent Application Publication
(10) Pub. No.: US 2018/0243293 A1
(43) Pub. Date: Aug. 30, 2018

(54) Pharmaceutical combinations and their use

(71) Applicant: Novartis AG, Basel (CH)

(72) Inventors: Ensar Halilovic, Quincy, MA (US); Caroline Emery, Kansas City, MO (US)

(21) Appl. No.: 15/751,954

(22) PCT Filed: Aug. 11, 2016
(86) PCT No.: PCT/IB2016/054841

§ 371 Date: Feb. 12, 2018

Related U.S. Application Data
(60) Provisional application No. 62/205,033, filed on Aug. 14, 2015.

(51) Int. Cl. A61K 31/496; A61K 31/506; A61K 31/517; A61K 31/497; A61P 35/00

(52) U.S. Cl. CPC A61K 31/496; A61K 31/506; A61P 35/00; A61K 31/497; A61K 31/517

(57) ABSTRACT

The present disclosure relates to a pharmaceutical combination, e.g. a product, comprising a combination of (a) a MDM2 inhibitor of formula I or formula II, or a pharmaceutically acceptable salt thereof and a PKC pathway inhibitor of formula III, formula IV, formula V or formula VI or a pharmaceutically acceptable salt thereof, particularly for use in the treatment or prevention of proliferative diseases. The disclosure also relates to corresponding pharmaceutical formulations, uses, methods, combinations, data carriers and related disclosure embodiments. The disclosure further relates to use of an MDM2 inhibitor of formula I or formula II, or a pharmaceutically acceptable salt thereof, alone for use in the treatment of a proliferative disease.

### FIELD OF THE DISCLOSURE

The present disclosure relates to a pharmaceutical combination comprising two targeted therapies, namely an MDM2 inhibitor and a protein kinase C (PKC) inhibitor, for use in the treatment or prevention of proliferative diseases. The disclosure also relates to corresponding pharmaceutical formulations, uses, methods, combinations, data carriers and related disclosure embodiments. The disclosure further relates to use of an MDM2 inhibitor of formula I or formula II, or a pharmaceutically acceptable salt thereof, alone for use in the treatment of a proliferative disease.

### BACKGROUND OF THE DISCLOSURE

Uveal melanoma (UM) is the most common cancer of the eye in adults (Singh A D. et al., Ophthalmology. 2011; 118: 1881-5). Most UM patients develop metastases for which no curative treatment has been identified so far.

The majority of UM tumors have mutations in the genes GNAQ (guanine nucleotide-binding protein G(q) subunit alpha) and GNA11 (guanine nucleotide-binding protein G(q) subunit 11), which encode for small GTPases (Harbour J W. Pigment Cell Melanoma Res. 2012; 25:171-81). Both of these mutations lead to activation of the protein kinase C (PKC) pathway. The up-regulation of PKC pathway has downstream effects which leads to constitutive activation of the mitogen-activated protein kinase (MAPK) signaling pathway that has been implicated in causing uncontrolled cell growth in a number of proliferative diseases.

Whilst anti-proliferative effects have been observed with certain PKC pathway inhibitors, no sustained MAPK pathway inhibition has been observed. Thus far, PKC inhibitors (PKCi) have had limited efficacy as single agents in patients (Mochly-Rosen D et al., Nat Rev Drug Discov. 2012 December; 11(12):937-57). Moreover, inhibition of PKC alone was unable to trigger cell death in vitro and/or tumor regression in vivo (Chen X, et al., Oncogene. 2014; 33:4724-34).

The protein p53 is a transcription factor that controls the expression of a multitude of target genes involved in DNA damage repair, apoptosis and cell cycle arrest, which are all important phenomena counteracting the malignant growth of tumors. The TP53 gene is one of the most frequently mutated genes in human cancers, with approximately half of all cancers having inactivated p53. Furthermore, in cancers with a non-mutated TP53 gene, typically the p53 is functionally inactivated at the protein level. One of the mechanisms of p53 inactivation is through its interaction with human homolog of MDM2 (Mouse double minute 2) protein. MDM2 protein functions both as an E3 ubiquitin ligase, that leads to proteasomal degradation of p53, and an inhibitor of p53 transcriptional activation. Therefore, MDM2 is an important negative regulator of the p53 tumor suppressor. MDM2 inhibitors can prevent interaction between MDM2 and p53 and thus allow the p53 protein to exert its effector functions. Whilst TP53 mutations are not common in UM, there are reports suggesting the p53 pathway is inactivated by either high expression of MDM2 protein or downregulation of the PERP protein in UM patients.

A combination of an MDM2 inhibitor (Nutlin-3) has been shown to act synergistically with reactivation of p53 and induction of tumor cell apoptosis (RITA) and Topotecan to cause growth inhibition in UM cell lines (De Lange J. et al., Oncogene. 2012; 31:1105-16). However, Nutlin-3 and Topotecan delayed in vivo tumor growth only in a limited manner.

### SUMMARY OF THE INVENTION

The following disclosure pertains to dually targeting p53, either alone or in combination with the PKC pathway in order to treat UM. In this way the MDM2 inhibitor promotes the beneficial effect of another compound that targets a possibly subordinate, interdependent or simply coexisting biochemical pathway implicated in causing a proliferative disease.

Therefore, a pharmaceutical composition comprising at least (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, or (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one or a pharmaceutically acceptable salt thereof, optionally further comprising 3-(1.H.-indol-3-yl)-4-[2-(4-methyl-piperazin-1-yl)-quinazolin-4-yl]-pyrrole-2,5-dione, or a pharmaceutically acceptable salt thereof, 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof, 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof, or 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof, is provided.

Specifically, the present disclosure provides the following aspects, advantageous features and specific embodiments, respectively alone or in combination, as listed in the following items:
1. A mouse double minute 2 inhibitor (MDM2i), selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, for use in the treatment of uveal melanoma.
2. The MDM2i according to item 1, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof.
3. The MDM2i according to item 1, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.
4. A pharmaceutical combination comprising:
   (i) a MDM2i, selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof; and,
   (ii) at least one protein kinase C pathway inhibitor (PKCi) selected from 3-(1.H.-indol-3-yl)-4-[2-(4-methyl-piperazin-1-yl)-quinazolin-4-yl]-pyrrole-2,5-dione, or a pharmaceutically acceptable salt thereof; 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof;
   3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof; and 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.
5. The pharmaceutical combination according to item 4 for simultaneous, separate or sequential use.
   The pharmaceutical combination according to item 4 or item 5, further comprising at least one pharmaceutically acceptable carrier.
6. The pharmaceutical combination according to any one of items 4 to 6 in the form of a fixed combination.
7. The pharmaceutical combination according to any one of items 4 to 7 in the form of a kit of parts for combined administration, wherein the MDM2i and the PKCi are administered independently at the same time or separately within time intervals, especially where these time intervals allow the combination partners to be jointly therapeutically active.
8. The pharmaceutical combination according to any one of items 4 to 8 wherein the MDM2i and PKCi are in a quantity which is jointly therapeutically effective for the treatment of uveal melanoma.
9. The pharmaceutical combination according to any one of items 4 to 9 in the form of a combination product.
10. The pharmaceutical combination according to any one of items 4 to 10 for use in the treatment of uveal melanoma.
11. A MDM2i according to any one of items 1 to 3, or the pharmaceutical combination according to item 9 or item 11, wherein the uveal melanoma is metastatic uveal melanoma.
12. A MDM2i according to any one of items 1 to 3, or the pharmaceutical combination according to item 9, item 11 or 12, wherein the uveal melanoma comprises metastasis of uveal melanoma.
13. A MDM2i according to any one of items 1 to 3, 11 or 12, or the pharmaceutical combination according to any one of items 9 or 11 to 13, wherein the uveal melanoma comprises functional p53 or wild-type TP53.
14. A pharmaceutical combination according to any one of items 9 or 11 to 14, wherein the uveal melanoma or metastatic uveal melanoma is characterized by mutation of guanine nucleotide-binding protein G(q) subunit alpha (GNAQ) gene or guanine nucleotide-binding protein G(q) subunit 11 (GNA11) gene.
15. Use of a data carrier comprising information about using the pharmaceutical combinations according to any one of items 4 to 15 simultaneously, separately or sequentially, and/or to instruct to administer the pharmaceutical combinations according to any one of items 4 to 15, simultaneously, separately or sequentially for the treatment of uveal melanoma.
16. A method of treating a patient suffering from uveal melanoma or metastatic uveal melanoma comprising administering to the patient either simultaneously, separately or sequentially the pharmaceutical combination according to any one of items 4 to 16 wherein the amount of the pharmaceutical combination is therapeutically effective in the treatment of uveal melanoma or metastatic uveal melanoma.
17. A MDM2i, selected from (i) (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and (ii) a PKCi selected from 3-(1.H.-indol-3-yl)-4-[2-(4-methyl-piperazin-1-yl)-quinazolin-4-yl]-pyrrole-2,5-dione, or a pharmaceutically acceptable salt thereof; 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof; 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof; and 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof, for combined use as a medicine.
18. The MDM2i according to item 18, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and the PKCi 3-(1.H.-indol-3-yl)-4-[2-(4-methyl-piperazin-1-yl)-quinazolin-4-yl]-pyrrole-2,5-dione, or a pharmaceutically acceptable salt thereof, for combined use as a medicine.
19. The MDM2i according to item 18, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and the PKCi is 3-(1.H.-indol-3-yl)-4-[2-(4-methyl-piperazin-1-yl)-quinazolin-4-yl]-pyrrole-2,5-dione, or a pharmaceutically acceptable salt thereof, for combined use as a medicine.
20. The MDM2i according to item 18, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and the PKCi is 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof, for combined use as a medicine.
21. The MDM2i according to item 18, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and the PKCi is 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof, for combined use as a medicine.
22. The MDM2i according to item 18, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and the PKCi is 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof, for combined use as a medicine.
23. The MDM2i according to item 18, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and the PKCi is 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof, for combined use as a medicine.
24. The MDM2i according to item 18, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and the PKCi is 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof, for combined use as a medicine.
25. The MDM2i according to item 18, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and the PKCi is 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof, for combined use as a medicine.
26. The pharmaceutical combination according to any one of items 4 to 14, use of a data carrier according to item 15, method of treating a patient according to item 16, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof.
27. The pharmaceutical combination according to any one of items 4 to 15, use of a data carrier according to item 16, method of treating a patient according to item 17, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.
28. The pharmaceutical combination according to any one of items 4 to 15, use of a data carrier according to item 16, method of treating a patient according to item 17, wherein the PKCi is 3-(1.H.-indol-3-yl)-4-[2-(4-methyl-piperazin-1-yl)-quinazolin-4-yl]-pyrrole-2,5-dione, or a pharmaceutically acceptable salt thereof.
29. The pharmaceutical combination according to any one of items 4 to 15, use of a data carrier according to item 16, method of treating a patient according to item 17, wherein the PKCi is 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof.
30. The pharmaceutical combination according to any one of items 4 to 15, use of a data carrier according to item 16, method of treating a patient according to item 17, wherein the PKCi is 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof.
31. The pharmaceutical combination according to any one of items 4 to 15, use of a data carrier according to item 16, method of treating a patient according to item 17, wherein the PKCi is 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.

### BRIEF DESCRIPTION OF FIGURES

FIG. 1: Co-inhibition of PKC and MDM2 induces cell death in the majority of UM cell lines. (A) Compound C (inhibition of PKC) and compound A (inhibition of MDM2) were used respectively at 500 nM and 1 μM final concentration. Growth curve under treatment with compound A or/and compound C. Cell viability was measured every 3 days with compound replacement at day 6. All cell lines contained GNAQ/11 mutations. Averages between triplicates are represented ±SEM. (B) Control cell lines without GNAQ/11 mutations.

FIG. 2: Co-inhibition of PKC and MDM2 induces cell death in the majority of UM cell lines. Molecular analyses by western blot. Apoptosis was assessed by cPARP. pMARCKS and pPKCd were used as pharmacodynamic markers for compound C activity, while p53 and p21 were used as the marker for compound A activity.

FIG. 3: In vitro evaluation of compound A and compound C combinations Histogram ranking all tested cell lines according to their synergy score. Right: Dot Plot representing Amax values (y-axis) and synergy scores (x-axis) for all tested cell lines.

FIG. 4: In vivo efficacy of compound A and compound C combination in the 5 UM PDXs. Tumor growth was evaluated by plotting the mean of the RTV (relative tumor volume)±SD per group.

FIG. 5: In vivo efficacy of compound A and compound C in the 5 UM PDXs. The overall response rate (ORR) of mice treated by compound A and compound C was defined as the relative tumor volume variation (RTVV) of each compound A- and compound C-treated mouse calculated from the following formula: [(Vt/Vc)−1], where Vt is the volume of the treated mouse and Vc the median volume of the corresponding control group at a time corresponding to the end of treatment

FIG. 6. Selected dose response curves for Compound B. Compound B was tested against different UM cell lines, the dose response curves for each are displayed.

FIG. 7. The in vitro effect on proliferation of combining the PKC inhibitor Compound C with the Mdm2 inhibitor Compound B in the UM 92.1 cell line. Shown here are matrices for inhibition of growth and Loewe (ADD) excess inhibition for compound B combinations with compound C in the UM cell lines. The combination resulted in synergistic anti-proliferative effects (synergy score=2.42).

FIG. 8. The in vitro effect on proliferation of combining the PKC inhibitor Compound D with the Mdm2 inhibitor Compound B in the UM 92.1 cell line. Shown here are matrices for inhibition of growth and Loewe (ADD) excess inhibition for compound B combinations with compound D in the UM cell lines. The combination resulted in synergistic anti-proliferative effects (synergy score=2.24).

### DETAILED DESCRIPTION OF THE DISCLOSURE

Based on successful experiments with sample compounds it has been determined that combining the specific MDM2 inhibitors (Mdm2i) with the PKC inhibitors (PKCi) can provide efficacious means to treat uveal melanoma. The present disclosure provides a specific MDM2 inhibitor (Mdm2i) for use in the treatment of uveal melanoma. The present disclosure also provides a pharmaceutical combination comprising (i) an MDM2 inhibitor (Mdm2i) of formula I or formula II, or a pharmaceutically acceptable salt thereof and (ii) a PKC inhibitor of formula III, formula IV, formula V, formula VI or a pharmaceutically acceptable salt thereof.

The present disclosure relates to compounds that exhibit anti-proliferative activity when used alone and in combination, preferably in UM patients. Suitably, the method relates to methods of treating a proliferative disease by administration or co-administration of said compounds.

The present disclosure provides a pharmaceutical combination comprising (i) an MDM2 inhibitor of formula I or formula II, or a pharmaceutically acceptable salt thereof and (ii) a PKC inhibitor (PKCi) of formula III, formula IV, formula V, formula VI or a pharmaceutically acceptable salt thereof

By targeting p53, either alone or in combination with the PKC pathway, the pharmaceutical compositions and pharmaceutical combinations provided herein have been surprisingly found to be useful in treating UM or metastatic UM. The pharmaceutical compositions and pharmaceutical combinations and/or drug regimens described herein led to the induction of cell death in vitro, tumor stabilization and even tumor regression in vivo, with a surprisingly high in vivo tumor shrinkage observed in one combination.

The MDM2 inhibitor can be (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one (compound A) of formula I:

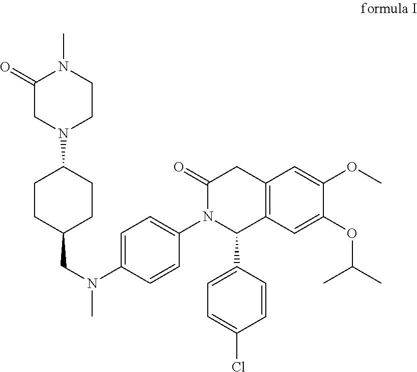 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

Compound A of formula I can be prepared as described in WO2011/076786.

The MDM2 inhibitor can also be (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (compound B) of formula II:

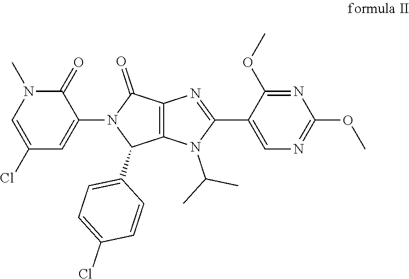 [SMILES: COc1ncc(-c2nc3c(n2C(C)C)[C@H](c2ccc(Cl)cc2)N(c2cc(Cl)cn(C)c2=O)C3=O)c(OC)n1]

The compound of formula II can be prepared as described in WO2013/111105 and is even the preferred compound to be used in the present pharmaceutical combination.

As a combination partner in the pharmaceutical combination of the present invention, the PKC inhibitor can be 3-(1.H.-indol-3-yl)-4-[2-(4-methyl-piperazin-1-yl)-quinazolin-4-yl]-pyrrole-2,5-dione (compound C) of formula III:

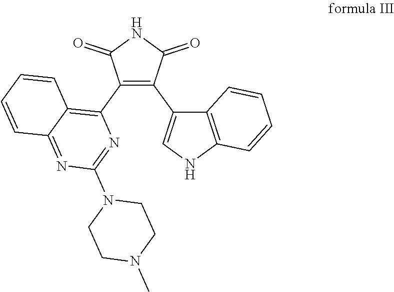 [SMILES: CN1CCN(c2nc(C3=C(c4c[nH]c5ccccc45)C(=O)NC3=O)c3ccccc3n2)CC1]

The compound of formula III can be prepared as described in WO02/38561.

The PKC inhibitor as used herein can also be 3-amino-N-(3-(4-amino-4-methylpiperidin-1-yl)pyridin-2-yl)-6-(3-(trifluoromethyl)pyridin-2-yl)pyrazine-2-carboxamide (compound D) of formula IV:

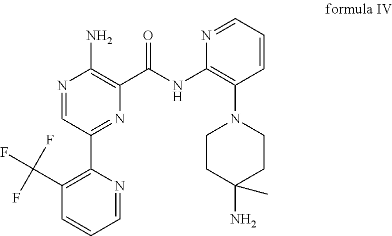 [SMILES: CC1(N)CCN(c2cccnc2NC(=O)c2nc(-c3ncccc3C(F)(F)F)cnc2N)CC1]

Another possible PKC inhibitor for use in the combination with the Mdm2i is 3-amino-N-(3-(4-aminopiperidin-1-yl)pyridin-2-yl)-6-(3-(trifluoromethoxy)pyridin-2-yl)pyrazine-2-carboxamide (compound E) of formula V:

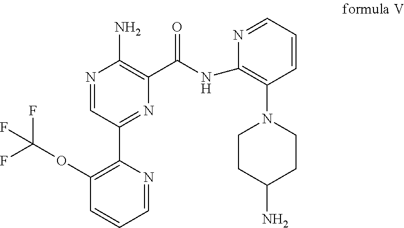 [SMILES: Nc1ncc(-c2ncccc2OC(F)(F)F)nc1C(=O)Nc1ncccc1N1CCC(N)CC1]

Another PKC inhibitor as used herein can also be 3-amino-N-(3-(4-amino-4-Methylpiperidin-1-yl)pyridin-2-yl)-6-(3-(trifluoromethoxy)pyridin-2-yl)pyrazine-2-carboxamide (compound F) of formula VI:

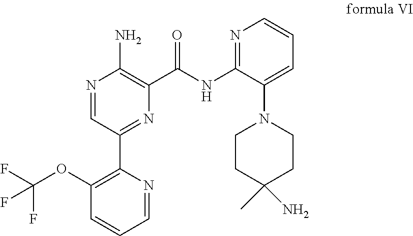 [SMILES: CC1(N)CCN(c2cccnc2NC(=O)c2nc(-c3ncccc3OC(F)(F)F)cnc2N)CC1]

Compounds of formula IV, V and VI can be prepared as described in international Application No. PCT/162015/055951.

The present disclosure encompasses embodiments that include all pharmaceutically acceptable salts of the compounds useful according to the disclosure provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. For example, the salt is sulphate salt, or bisulphate salt.

Compounds, particularly compound D, E and F (i.e. compounds of formulas IV, V, and VI, respectively) may be in a form of a pharmaceutically acceptable prodrug. The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the disclosure. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds described herein, e.g. a MDM2 inhibitor and/or a PKC inhibitor, are intended to be used in combination, especially for use in a pharmaceutical combination that may optionally include further co-agents as defined below. All of these materials may be referred to as “active ingredients” in the combination. It should be understood that both terms (e.g. compound(s) and active ingredient(s)) encompasses pharmaceutically acceptable salts, prodrugs, tautomers, N-oxides, or solvates, e.g. hydrates, of these materials. It should be understood when reading this disclosure that the combinations of the present application encompasses all the aforementioned variants, as well as any single one thereof or combination of two or more to less than all such variants.

The present disclosure provides a pharmaceutical combination comprising (i) an MDM2 inhibitor of formula I or formula II, or a pharmaceutically acceptable salt thereof and (ii) a PKC inhibitor of formula III, formula IV, formula V, formula VI or a pharmaceutically acceptable salt thereof for use in the treatment of a patient in need thereof. The pharmaceutical combination of the compounds described herein can be used in the treatment of a patient with uveal melanoma (UM). The uveal melanoma can also be metastatic UM. In alternative, the combination can also be used to target metastasis of UM. Particularly the combination is suitable for treatment of a patient with UM or metastatic UM, wherein the UM comprises functional p53 or wild-type TP53. Such protein or gene status of a cancer is expected to make a patient with said cancer even more responsive to the combination of the present disclosure. Equally, further improved effect of the combination is expected in uveal melanoma or metastatic uveal melanoma, including metastasis thereof, which is characterized by mutation in either GNAQ or GNA11 genes. In patients harboring both, the functional p53 or wild-type TP53 and mutation in either GNAQ or GNA11 genes, the clinical response is expected to be pronounced the most. Therefore, the pharmaceutical combination of the present disclosure is best suited for use in the treatment of a patient with UM or metastatic UM, including UM metastasis, wherein the UM comprises functional p53 or wild-type TP53 and is characterized by mutation in either GNAQ or GNA11 genes.

The present disclosure, relates also to a pharmaceutical combination, especially a pharmaceutical combination product, comprising one or more of the compounds described herein and at least one pharmaceutically acceptable carrier.

The term “pharmaceutical combination” as used herein means a product that results from the use or mixing or combining of more than one active ingredient. It should be understood that pharmaceutical combination as used herein includes both fixed and non-fixed combinations of the active ingredients. The term “fixed combination” means that the active ingredients, e.g. a compound of formula (I) and one or more combination partners, are administered to a patient simultaneously as a single entity or dosage form. The term in such case refers to a fixed dose combination in one unit dosage form (e.g., capsule, tablet, or sachet). The terms “non-fixed combination” or a “kit of parts” both mean that the active ingredients, e.g. a compound of the present disclosure and one or more combination partners and/or one or more co-agents, are administered or co-administered to a patient independently as separate entities either simultaneously, concurrently or sequentially with no specific time limits wherein such administration provides therapeutically effective levels of the two compounds in the body of the patient, especially where these time intervals allow that the combination partners show a cooperative, e.g. synergistic effect. The term “non-fixed combination” also applies to cocktail therapy, e.g. the administration of three or more active ingredients. The term “non-fixed combination” thus defines especially administration, use, composition or formulation in the sense that the compounds described herein can be dosed independently of each other, i.e. simultaneously or at different time points. It should be understood that the term “non-fixed combination” also encompasses the use of a single agent together with one or more fixed combination products with each independent formulation having distinct amounts of the active ingredients contained therein. It should be further understood that the combination products described herein as well as the term “non-fixed combinations” encompasses active ingredients (including the compounds described herein) where the combination partners are administered as entirely separate pharmaceutical dosage forms or as pharmaceutical formulations that are also sold independently of each other. Instructions for the use of the non-fixed combination are or may be provided in the packaging, e.g. leaflet or the like, or in other information that is provided to physicians and/or medical staff. The independent formulations or the parts of the formulation, products, or compositions, can then be administered simultaneously or chronologically staggered, that is the individual parts of the kit of parts can each be administered at different time points and/or with equal or different time intervals for any part of the kit of parts. Particularly, the time intervals for the dosing are chosen such that the effect on the treated disease with the combined use of the parts is larger/greater than the effect obtained by use of only one of the compounds I-IV; thus the compounds used in pharmaceutical combination described herein are jointly active. The ratio of the total amounts of a compound of formula I or II to a compound of formula III-VI to be administered as a pharmaceutical combination can be varied or adjusted in order to better accommodate the needs of a particular patient sub-population to be treated or the needs of the single patient, which can be due, for example, to age, sex, body weight, etc. of the patients.

The terms “co-administration” or “combined administration” or the like as utilized herein are meant to encompass the administration of one or more compounds described herein together with a selected combination partner to a single subject in need thereof (e.g. a patient or subject), and are intended to include treatment regimens in which the compounds are not necessarily administered by the same route of administration and/or at the same time.

The term “pharmaceutical composition” is defined herein to refer to a mixture or solution (what about emulsions?) containing at least one active ingredient or therapeutic agent to be administered to a warm-blooded animal, e.g., a mammal or human, in order to prevent or treat a particular disease or condition affecting the warm-blooded animal.

The term “kit of parts” is defined herein to refer to especially combination partners (i) and (ii) as defined above, i.e. (i) being a MDM2i, selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof, and (ii) being at least one protein kinase C pathway inhibitor (PKCi) selected from 3-(1.H.-indol-3-yl)-4-[2-(4-methyl-piperazin-1-yl)-quinazolin-4-yl]-pyrrole-2,5-dione, or a pharmaceutically acceptable salt thereof; 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof; 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof; and 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof, can be dosed independently or by use of different fixed combinations with distinguished amounts of the combination partners (i) and (ii), i.e., simultaneously or at different time points. The parts of the kit of parts can then e.g., be administered simultaneously or chronologically staggered, that is at different time points and with equal or different time intervals for any part of the kit of parts. The ratio of the total amounts of the combination partner (i) to the combination partner (ii) to be administered in the combined preparation can be varied, e.g., in order to cope with the needs of a patient sub-population to be treated or the needs of the single patient.

The combination partners I to VI in any disclosure embodiment are preferably formulated or used to be jointly (prophylactically or especially therapeutically) active. This means in particular that there is at least one beneficial effect, e.g. a mutual enhancing of the effect of the combination partners I to VI, in particular a synergism, e.g. a more than additive effect, additional advantageous effects (e.g. a further therapeutic effect not found for any of the single compounds), less side effects, a combined therapeutic effect in a non-effective dosage of one or both of the combination partners I to VI, and very preferably a clear synergism of the combination partners I to VI.

The term “jointly therapeutically active” or “joint therapeutic effect” means that when the therapeutic agents, e.g. the active ingredients, are administered either in a chronologically staggered manner, especially a sequence-specific manner at preferred time intervals, in a warm-blooded animal, especially a human, to be treated, show a preferably synergistic interaction (joint therapeutic effect). Whether this is the case can, inter alia, be determined by following the blood levels, showing that both compounds are present in the blood of the human to be treated at least during certain time intervals.

As used herein, the term “patient” or “subject” refers to an animal. Typically the animal is a mammal. A patient also refers to for example, primates (e.g., humans), cows, sheep, goats, horses, dogs, cats, rabbits, rats, mice, fish, birds and the like. In certain embodiments, the patient is a primate. In yet other embodiments, the patient is a human.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical combination product according to the disclosure (as a fixed combination, or non-fixed combination or as a kit of parts, e.g. as a combination of a fixed combination and/or individual formulations for one or both combination partners or as kit of individual formulations of the combination partners) comprises the combination of the present disclosure and one or more pharmaceutically acceptable carrier materials (carriers, excipients). The pharmaceutical combination or the combination partners constituting it can be formulated for particular routes of administration such as oral administration, parenteral administration, and rectal administration, etc. In addition, the combination products of the present disclosure can be made up in a solid form (including without limitation capsules, tablets, pills, granules, powders or suppositories), or in a liquid form (including without limitation solutions, suspensions or emulsions). The combination products and/or their combination partners (compounds, active ingredients) can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

The present disclosure thus pertains to a combination product for simultaneous or sequential use, such as a combined preparation or a pharmaceutical fixed combination, or a combination of such preparation and combination.

In the combination therapies of the disclosure, the compounds useful according to the disclosure may be manufactured and/or formulated by the same or different manufacturers. Moreover, the combination partners may be brought together into a combination therapy: (i) prior to release of the combination product to physicians (e.g. in the case of a kit comprising the compound of the disclosure and the other therapeutic agent); (ii) by the physician themselves (or under the guidance of a physician) shortly before administration; (iii) in the patient themselves, e.g. during sequential administration of the compound of the disclosure and the other therapeutic agent.

The information about the present combination or the use thereof in the treatment of uveal melanoma as described above and below can be shown on a data carrier, such as for example a product information leaflet, a summary of product characteristics, a brochure, marketing material, a web page, or when such information is stored or used on a data carrier such as for example a computer, an USB stick or a CD. Data carrier comprising information about using (i) an MDM2i of formula I or formula II, or a pharmaceutically acceptable salt thereof, and (ii) a PKCi, of formula III, formula IV of formula V or formula VI or a pharmaceutically acceptable salt thereof, simultaneously or sequentially, is disclosed. The data carrier, for example in a form of a product information leaflet or a label, packaging, brochure or web page instruction can be used to instruct to administer (i) a MDM2i of formula I or formula II, or a pharmaceutically acceptable salt thereof, and (ii) a PKCi, of formula III, formula IV of formula V or formula VI or a pharmaceutically acceptable salt thereof, simultaneously or sequentially for the treatment of cancer. The data carrier is particularly useful in the event the two partners of the combination are not formulated together, and supplied or sold separately. Each of the partners can be supplied with the data carrier, or even have the data carrier detached or provided separately, that informs or instructs about the possibility to use the combination partner in a pharmaceutical combination of the present disclosure. The data carrier can be used for the same purpose also in fixed combinations or situations, where both partners are supplied or sold together.

In certain embodiments, any of the above pharmaceutical combination, use, administration, composition, method, product or formulation involves further administering one or more other (e.g. third) co-agents.

Thus, the disclosure relates in a further embodiment to a pharmaceutical combination, particularly a pharmaceutical composition or a product comprising a therapeutically effective amount of (i) a MDM2i and (ii) a PKCi, or a pharmaceutically acceptable salt thereof, respectively, and at least one third therapeutically active agent (herein referred to as an “additional co-agent”), e.g. another active ingredient. The additional co-agent is preferably selected from the group consisting of an anti-cancer agent and an anti-inflammatory agent, particularly is an anti-cancer agent.

The combination partners (e.g. the individual compounds described herein) that together form a corresponding pharmaceutical combination according to the disclosure may be mixed to form a fixed pharmaceutical composition or they may be administered separately or at approximately the same time (i.e. before, simultaneously with or after the other drug substance(s)).

The pharmaceutical compositions that comprise the pharmaceutical combination of the application can be tablets or gelatin capsules comprising the active ingredient together with one or more commonly known carriers, e.g. one or more carriers selected from the group consisting of
a) Diluents, e.g., lactose, dextrose, sucrose, mannitol, sorbitol, cellulose and/or glycine;
b) Lubricants, e.g., silica, talcum, stearic acid, its magnesium or calcium salt and/or polyethyleneglycol; for tablets also
c) Binders, e.g., magnesium aluminum silicate, starch paste, gelatin, tragacanth, methylcellulose, sodium carboxymethylcellulose and/or polyvinylpyrrolidone; if desired
d) Disintegrants, e.g., starches, agar, alginic acid or its sodium salt, or effervescent mixtures; and
e) Absorbents, colorants, flavors and sweeteners.

Tablets may be either film coated or enteric coated according to methods known in the art.

Suitable compositions for oral administration especially include an effective amount of one or more or in case of fixed combination formulations each of the combination partners (active ingredients) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient(s) in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Doses of Mdm2 inhibitors used in a composition may vary and is dependent for example on the route of administration, gender of a patient, weight, stadium of a disease, etc.

Parenteral compositions and other can be prepared by known methods in the art.

The following Examples illustrate the disclosure and provide specific embodiments, however without limiting the scope of the disclosure.

### EXAMPLES

### Example 1: Monotherapy of Compound A and Compound C and Combination Therapy of Compound A with Compound C

### Uveal Melanoma Preclinical Models

Five PDXs representative of the UM disease were used: MP42, MP46, MP55, MM33 and MM52 (Table 1). The main molecular features of these PDXs have been described in Table 1.

To corroborate in vivo findings, fifteen cellular models, isolated either from primary tumors or metastases, were used in this study MP38, MP41, MP46, MP65, MM28 and MM66 cell lines were established in our laboratory as described in. 92.1 and Mel202 cell lines were purchased from The European Searchable Tumour Line Database (Tubingen University, Germany), and MRC5 and RPE1 lines from ATCC. OMM1, OMM2.5, Mel285 and Mel290 cells were kindly provided by P. A. Van Der Velden (Leiden University, The Netherlands). Cell lines were cultured in RPMI-1640 supplemented with 10% FBS (92.1, Mel202, OMM1, OMM2.5, Mel285, Mel290, MRC5, RPE1) or 20% FBS (MP38, MP41, MP46, MP65, MM28, MM66), complemented with Penicillin at 100 U/ml and Streptomycin 100 μg/ml (Life Technologies). The two primary cultures of normal melanocytes isolated from a human choroid were kindly given by G. Liot (Institut Curie, France). These cells were cultured in Ham/F12 medium supplemented with 10% FBS, Penicillin/streptavidin, FGF2 at 10 ng/ml, IBMX at 0.1 mM and cholera toxin at 10 ng/ml. IBMX and cholera toxin were removed from the medium during drug testing to avoid interference with PKC pathway. These cultures were sequenced to validate their melanocytic origin and the absence of GNAQ/11 mutation. All cells were proved to be Mycoplasma free and maintained at 37° C. in a humidified atmosphere with 5% CO2.

### Compounds

Compound A: (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one (MDM2 inhibitor)

Compound B: (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (MDM2 inhibitor)

Compound C: 3-(1.H.-indol-3-yl)-4-[2-(4-methyl-piperazin-1-yl)-quinazolin-4-yl]-pyrrole-2,5-dione (PKC inhibitor)

Compound D: 3-amino-N-(3-(4-amino-4-methylpiperidin-1-yl)pyridin-2-yl)-6-(3-(trifluoromethyl)pyridin-2-yl)pyrazine-2-carboxamide (PKC inhibitor)

All drugs used in this study (compound A and compound C) were obtained from Novartis Institutes for Biomedical Research (NIBR, Cambridge, USA). Compound C is a selective inhibitor of the classical (α, β) protein kinase C (PKC) that also has activity against novel (δ, ε, η, θ) PKC isoforms. Compound A is a selective inhibitor of MDM2. For in vivo administration, all compounds were diluted in 20% propylene glycol+50% solutol (20%)+30% PBS. The control groups were treated with this solution (vehicle). Compound C was administered per os twice a day, 5 days/week at a daily dose of 120 or 240 mg/kg according to the in vivo experiment design. Compound A was administered per os, 5 days/week at a daily dose of 100 mg/kg.

For in vitro experiments, compound powders were dissolved in DMSO at 10 mM final, aliquoted and stored at −20° C. Further dilutions were made according to each experiment design.

### In Vivo Drug Testing Experiments

Four to six week-old SCID mice, bred at Institut Curie, were used. Tumor fragments of 30-60 mm³ were grafted subcutaneously into the interscapular fat pad. When tumors reached a size of about 50-150 mm³, mice were randomly assigned to control or treatment groups. Between six to nine mice per group were included in each experiment. Xenografted mice were sacrificed when their tumor reached a volume of 2500 mm³.

Tumor growth was evaluated by measuring with a caliper two perpendicular tumor diameters twice a week. Individual tumor volume, relative tumor volume (RTV) and tumor growth inhibition (TGI) were calculated according to a standard method. Tumor stability or shrinkage was defined as a RTV≤1 at the end of experiments. To evaluate the response to each compound and combination according to individual mouse variability, we have considered each mouse as one tumor-bearing entity. We have defined a relative tumor volume variation (RTVV) of each treated mouse: RTVV=Vt/Vc, where Vt is the volume of the treated mouse and Vc the median tumor volume of the corresponding control group at the end of treatment. For each mouse, we calculated an overall response rate (ORR) using the formula: ORR=[(RTVV)−1]. A tumor was considered as responding to therapy when ORR was lower than −0.5. Since data were normalized to each control group, results of independent in vivo experiments could be merged.

Studies have been performed in compliance with the recommendations of the French Ethical Committee and under the supervision of authorized investigators. The experimental protocol and animal housing followed institutional guidelines as put forth by the French Ethical Committee (Agreement C75-05-18, France) and the ethics committee of Institut Curie.

### Statistical Tests for In Vivo Experiments

Two by two comparison of the TGI was done using a two-tailed Mann-Whitney test based on the RTVV. For all pairwise comparisons based on the proportions of tumors with a particular RTV or ORR, a two-tailed Fisher's exact test was used. All statistical tests were realized bilaterally calculating two-tailed p values. Results were considered statistically significant when p≤0.05 (95% confidence interval).

### Drug Combination Cell Viability Assay

Cells were seeded at appropriate concentration in 96-well plates following a 6×6 matrix design. Three plates were prepared per cell line to generate triplicates. The day after, each drug was added following a matrix dilution format. 1:3 serial dilutions were tested to result in a total of six serial dilutions, including the DMSO control. The highest drug concentration for each compound was adjusted so that the final concentrations of the two drugs had their full efficacy in monotherapy within the two highest doses. Cell viability was measured after five days of drug treatment using the MTT assay (Sigma). Colorimetric results were read using a spectrophotometer. Results are expressed as relative percentages of metabolically inactive cells compared with DMSO-treated controls (percentage of growth inhibition). All different combinations were tested on the whole panel of cell lines for each experimental procedure. The tests were repeated until at least an independent duplicate for each drug combination was obtained.

### Evaluation of In Vitro Combination Activity

We used data obtained with the Loewe algorithm, which calculates a weighted “Synergy Score” across the dose matrix that adjusts for dose sampling and coverage and weights to favor combination effects at high inhibition levels (Lehar et al. 2009). Synergy score and isobolograms were generated to quantify the combination strength. A synergy score higher than 2 was considered as significant when compared to the variation of synergy scores seen within self-crosses (drug-with-self; theoretical synergy score of 0) (Lehar et al. 2009).

### Cell Proliferation Assay and Evaluation of Cell Death

At day 0, cells were plated in triplicate at appropriate concentration in 96-well plates. Four conditions were tested for each cell line: DMSO, Drug A, Drug C and Drug A+C. At day 1, each drug was added to each well. Optimal drug concentrations were chosen from the combination experiments: Compound C was used at 500 nM, and compound A at 1 μM. The amount of DMSO was adjusted in each mix to get the same percentage of DMSO for each treatment condition. Compounds were replenished at day 6. At day 3, day 6 and day 9, viability was measured using the CellTiterGlo assay (Promega). Luminescence was read using a spectrophotometer. The average between triplicates was made and represented ±SEM. All cell lines were tested at the same time and at least two independent experiments were performed to confirm results reproducibility.

### Western Blot Analyses

Cells were cultured in 10 cm-diameter dishes and treated with DMSO or each drug as single agent or combination for 72 h. Western blot analyses were performed using standard procedures. GAPDH was used for normalization between samples. Primary antibodies were diluted in TBST+0.5% BSA at an appropriate dilution and incubated overnight at 4° C. All antibodies used in this study are listed in Supplementary Materials. Signal was detected using secondary antibodies coupled with HRP (Jackson laboratory). Luminescent signal was detected using a LAS-3000 Luminescent Image analyzer and Image Gauge software.

TABLE 1
Genetic status of the known altered genes in UM for all cell lines used in the study.

| | Cell line | | GNAQ | GNA11 | BAP1 | BAP1 | SF3B1 | eIF1AX | Additional |
|---|---|---|---|---|---|---|---|---|---|
| Tissue | name | Tumor | mutation | mutation | mutation | loss | mutation | mutation | comments |
| UM | MP38 | P | + | − | + | + | − | − | |
| | MP41 | P | − | + | − | − | − | − | |
| | MP46 | P | + | − | − | + | − | − | |
| | MP65 | P | − | + | + | + | − | − | |
| | MM28 | M | − | + | + | + | − | − | Liver |
| | | | | | | | | | metastasis |
| | MM66 | M | − | + | − | − | − | − | Liver |
| | | | | | | | | | metastasis |
| | 92.1 | P | + | − | − | − | − | + | |
| | Mel202 | P | + | − | − | − | + | − | |
| | Mel270 | P | + | − | − | − | − | − | |
| | OMM1 | M | − | + | − | − | − | − | Subcutaneous |
| | | | | | | | | | metastasis |
| | OMM2.5 | M | + | − | − | − | − | − | Liver |
| | | | | | | | | | metastasis |
| | Mel285 | P | − | − | − | − | − | − | |
| | Mel290 | P | − | − | − | − | − | − | |
| Normal | RPE1 | N/A | − | − | − | − | − | − | Immortalized |
| retina | | | | | | | | | with |
| | | | | | | | | | hTERT |
| Normal | MRC5 | N/A | − | − | − | − | − | − | fibroblasts |
| lung | | | | | | | | | |

P (primary tumor),
M (metastasis),
− (wild type),
+ (mutant),
N/A (non-applicable)

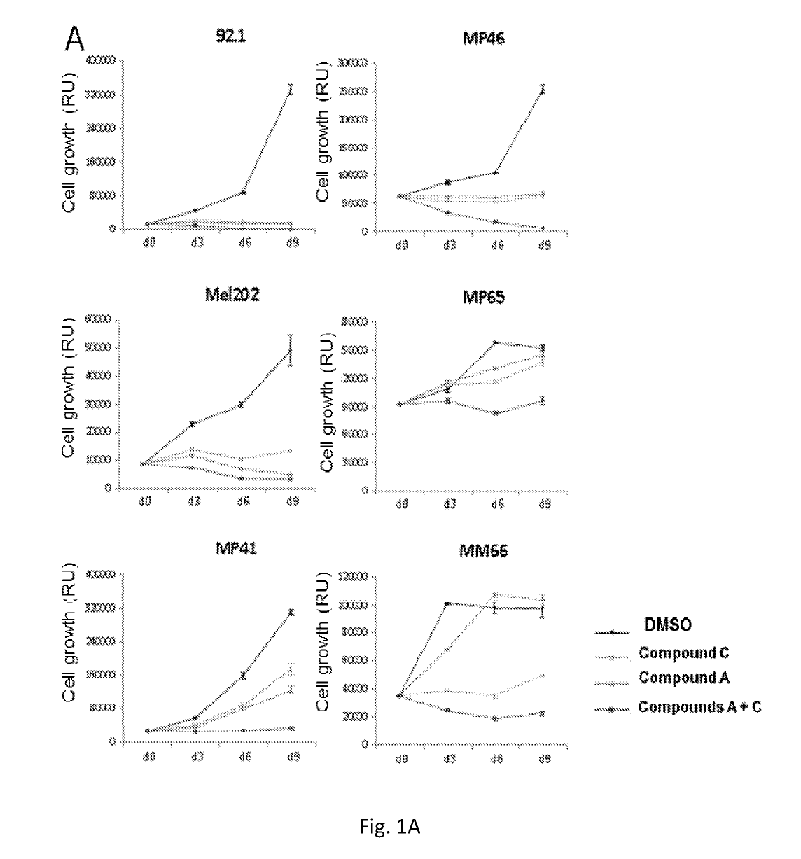

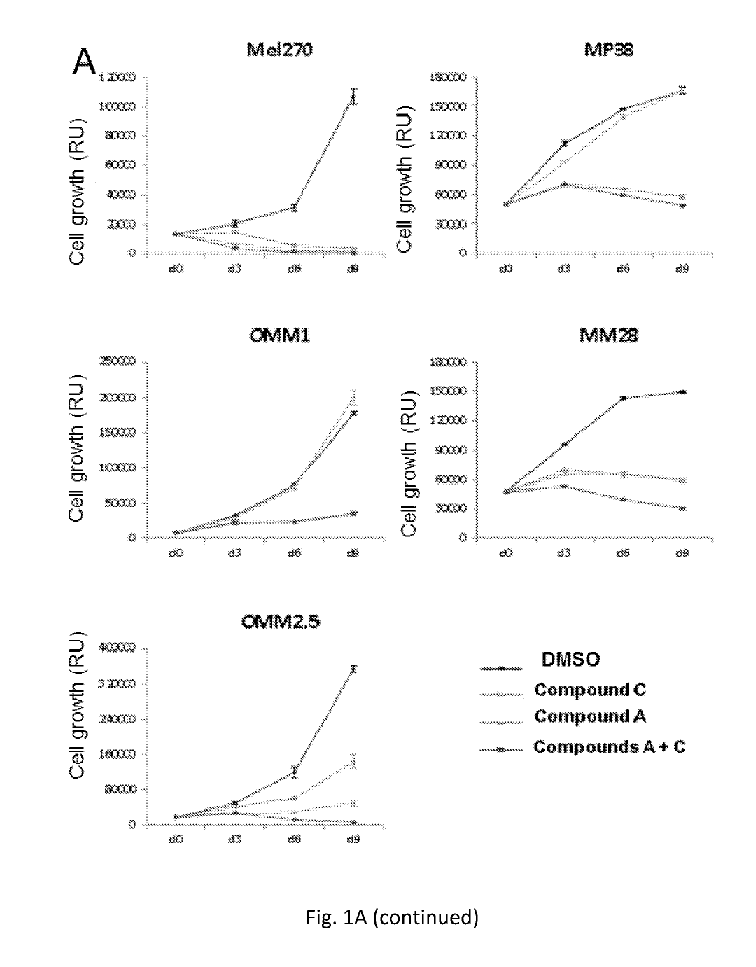

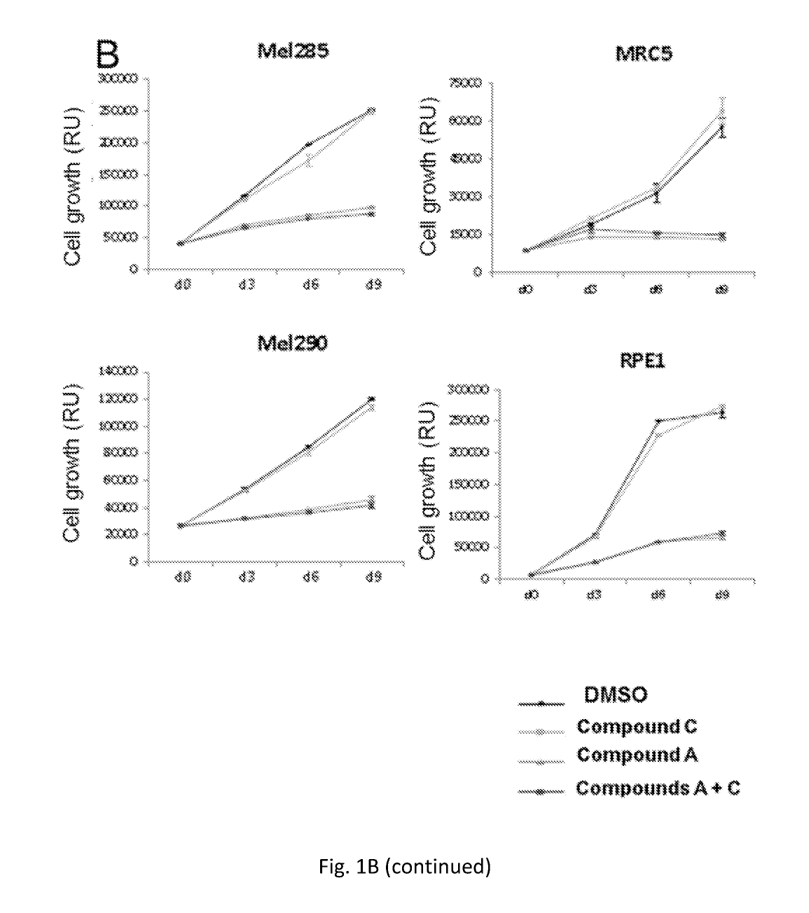

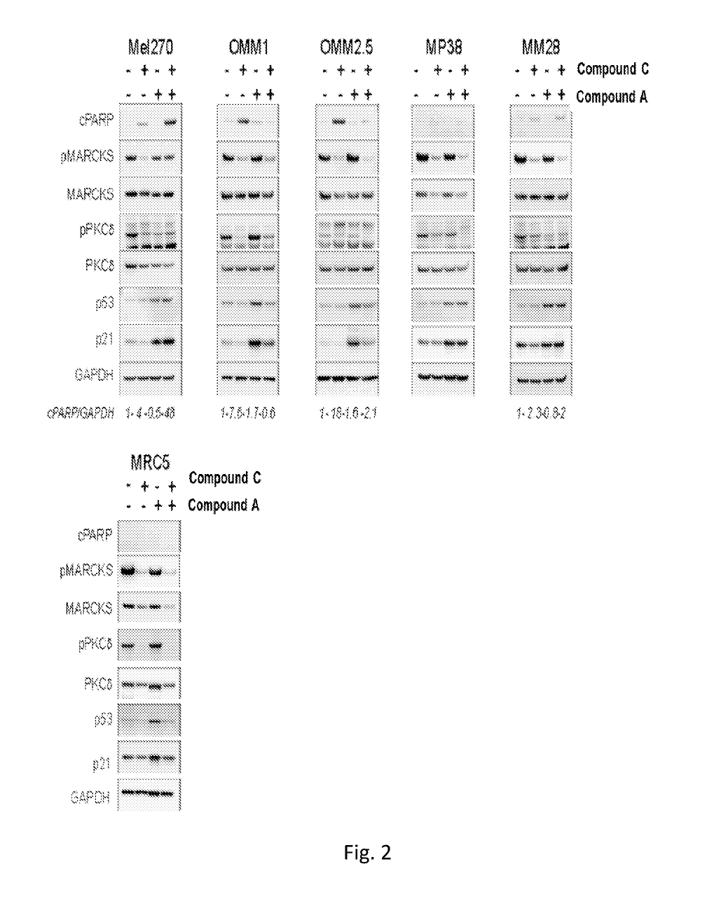

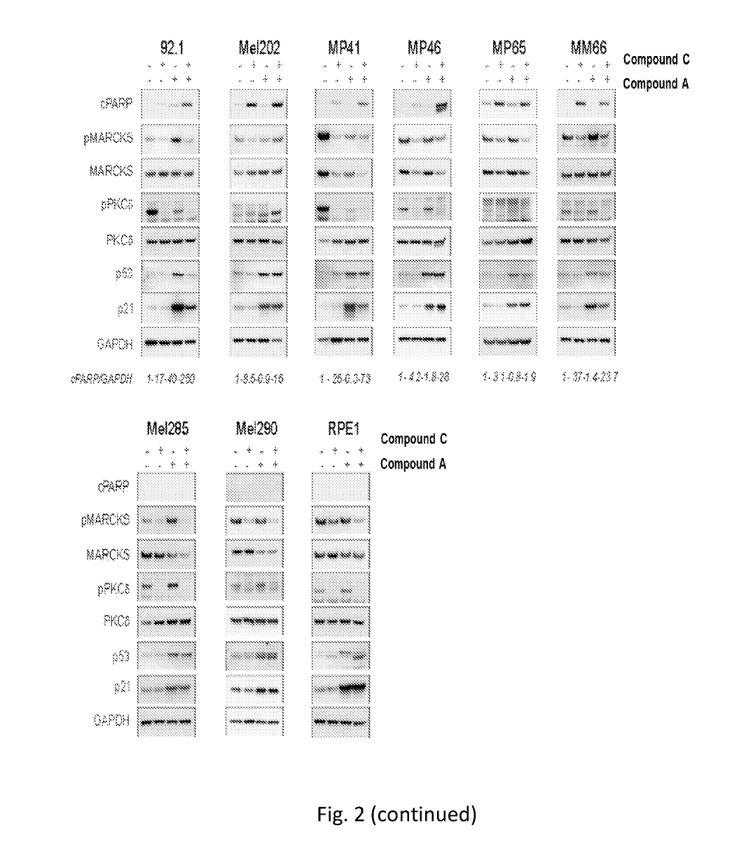

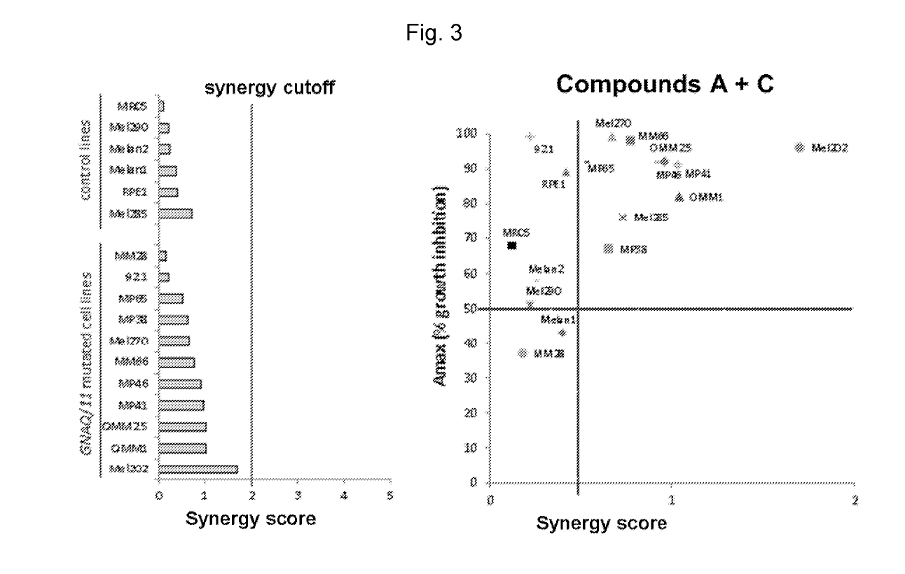

The results confirmed the combination activities between compound A and compound C indicating that co-inhibition of PKC and MDM2 have valuable therapeutic approaches for UM patients carrying GNAQ/11 mutations (FIGS. 1-5).

In vitro findings showed that co-inhibition of PKC and MDM2 are effective combination strategies for GNAQ/11 mutated UM models. Both co-treatments led to induction of apoptosis in more than 80% of the cellular models tested (FIGS. 1-3).

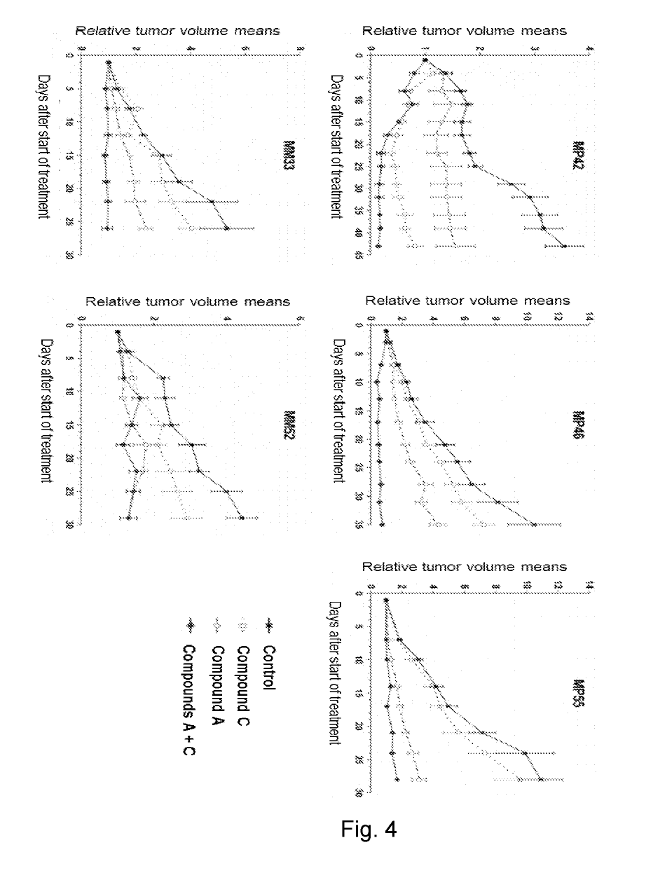

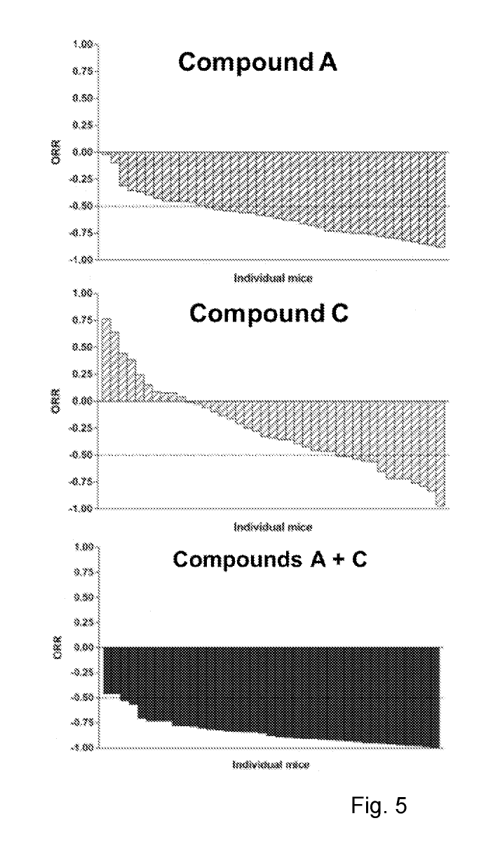

In vivo findings showed that co-inhibitions of PKC and MDM2 were effective combination strategies for GNAQ/11 mutated UM PDXs, with peculiarly strong tumor shrinkage after PKC and MDM2 inhibition (FIGS. 4-5).

### Example 2: Monotherapy of Compound B or Compound A and Combination Therapy of Compound B with Compound C or Compound D

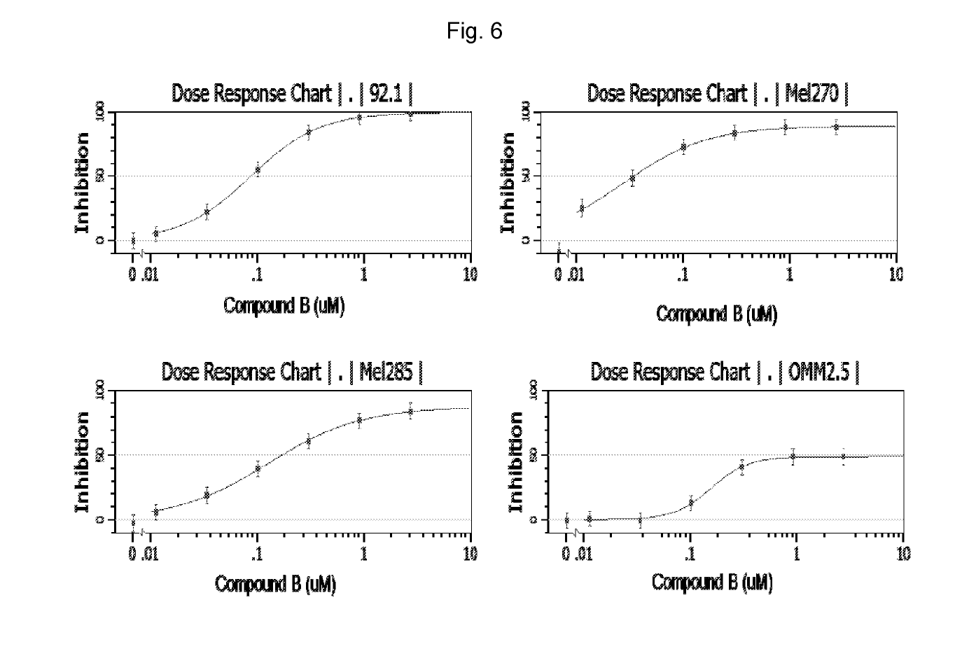

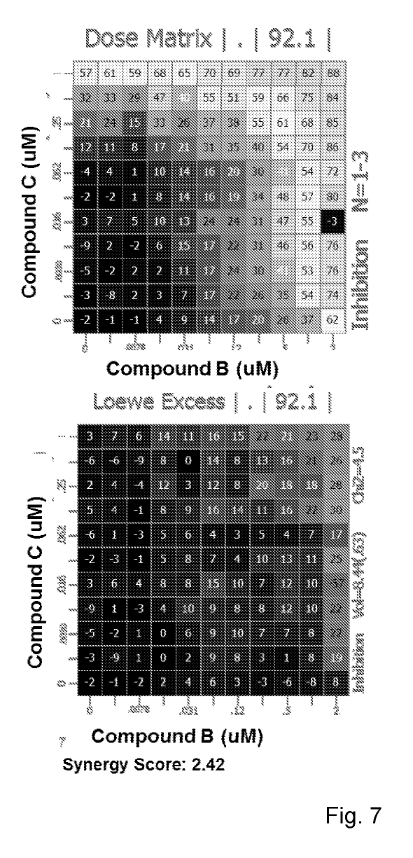

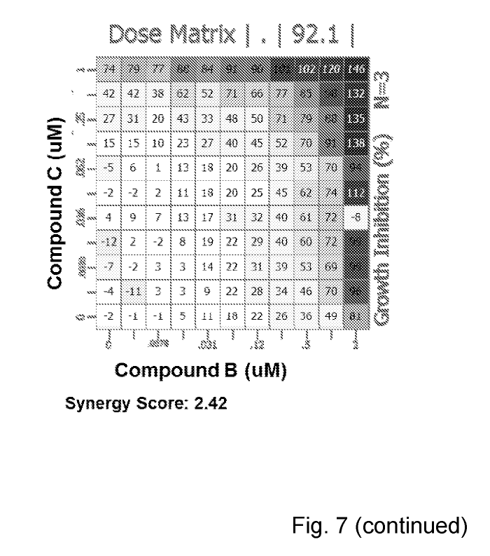

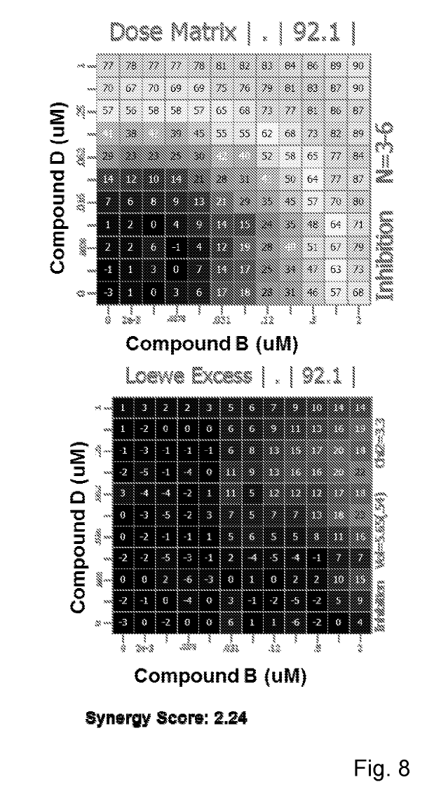

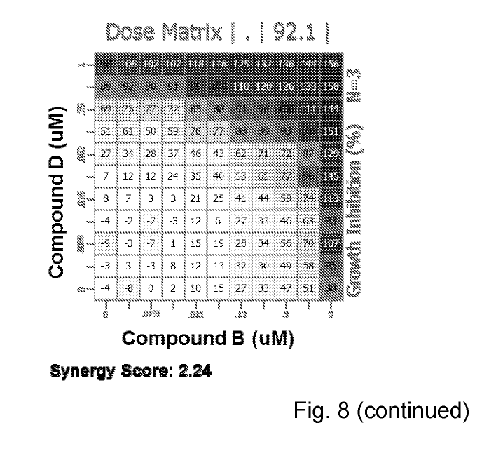

We tested efficacy of MDM2 inhibitors in total of six UM cell lines (FIG. 6). Efficacy of the combination of MDM2 and PKC inhibitors were tested in one UM cell line. Compound B as single agent strongly inhibited the growth of 5 out of the six cell lines evident by nanomolar IC50 values (Table 2). It is worth noting that both PKC inhibitors behave similarly in the combination with Compound B, however Compound D was more effective as a single agent. Synergy was calculated using the Loewe model as described in (Lehar et al. 2009), with a score above 2 being indicative of synergy (FIGS. 7 and 8).

TABLE 2
IC50 single agent activities of compounds A and B across
multiple UM cell lines
IC50 Values (μM)

| | Cell Line | | | | |
|---|---|---|---|---|---|
| | 92.1 | Mel202 | Mel270 | Mel285 | OMM2.5 |
| Compound A | 0.667 | 0.401 | 0.362 | 1.26 | 2.57 |
| Compound B | 0.086 | 0.060 | 0.034 | 0.167 | 0.249 |

### Methods

All compounds were dissolved in 100% DMSO (Sigma, Catalog number D2650) at concentrations of 10 mM and stored at −20° C. until use. Compounds were arrayed in 300 ul deep 384-well plates (BrandTech, catalogue number 701355) serially diluted 2-fold at 4× concentration. Then, 10 uL of the 4× compound plate was stamped onto 30 uL of cells resulting in a final 1× concentration. COMPOUND B was used over a concentration range of 0.0-2 uM. COMPOUND C was used over a concentration range of 0.0-1 uM, and COMPOUND D was used over a concentration range of 0.0-1 uM.

Cell lines were cultured in 37° C. and 5% CO2 incubator and expanded in T-75 flasks. In all cases cells were thawed from frozen stocks, expanded through passage using 1:3 dilutions, counted and assessed for viability using a ViCell counter (Beckman-Coulter), prior to plating in 384-well. To split and expand cell lines, cells were dislodged from flasks using 0.25% Trypsin-EDTA (GIBCO, Catalogue number 25200). All cell lines were determined to be free of mycoplasma contamination as determined by a PCR detection methodology performed at Idexx Radii (Columbia, Mo., USA) and correctly identified by detection of a panel of SNPs.

Cell proliferation was measured in 72 hr CellTiter-Glo™ (CTG) assays and all results shown are the result of at least triplicate measurements. For CellTiter-Glo™ assays, cells were dispensed into tissue culture treated 384-well plates (Costar, catalogue number 3707) with a final volume of 30 μL of medium and at density of 1000 cells per well. 12 to 24 hrs after plating, 10 μL of each compound dilution series were transferred to plates containing the cells, resulting in compound concentration ranges stated above and a final DMSO concentration of 0.16%. Plates were incubated for 72 hrs and the effects of compounds on cell proliferation was determined using the CellTiter-Glo™ Luminescent Cell Viability Assay (Promega) and a Victor™ X4 plate reader (Perkin Elmer).

The CellTiter-Glo® Luminescent Cell Viability Assay is a homogeneous method to determine the number of viable cells in culture based on quantitation of the ATP present, which signals the presence of metabolically active cells. The method is described in detail in the Technical Bulletin, TB288 Promega. Briefly, cells were plated in Opaque-walled multiwell plates in culture medium as described above. Control wells containing medium without cells were also prepared to obtain a value for background luminescence. A volume of CellTiter-Glo® Reagent equal to the volume of cell culture medium present in each well was then added and contents mixed for 60 minutes on an orbital shaker to induce cell lysis. Next, luminescence was recorded using the plate reader.

The percent growth inhibition and excess inhibition were analysed using the Chalice software (CombinatoRx, Cambridge Mass.). The percentage of growth inhibition relative to DMSO is displayed in the panel labelled inhibition, and the amount of inhibition in excess of the expected amount in the panel (labelled ADD Excess Inhibition). Concentrations of COMPOUND C are shown along the bottom row from left to right and increasing concentrations of COMPOUND B along the leftmost column from bottom to top. All remaining points in the grids display results from a combination of the two inhibitors that correspond to the single agent concentrations denoted on the two axes. Data analysis of cell proliferation was performed using Chalice Analyser as described in (Lehar et al. 2009). Excess inhibition was calculated using the Loewe synergy model which measures the effect on growth relative to what would be expected if two drugs behave in a dose additive manner. Positive numbers represent areas of increasing synergy.

IC50 is the compound concentration which inhibits 50% of the CTG signal by 50%. IC50 calculations were made using model number 203 from the XLfit Microsoft Excel™ add-In version 5.2.0.0 (IDBS Enabling Science). Synergy scores and IC50 calculations were determined as described in (Lehar et al. 2009).

1.-32. (canceled)

33. A compound for the treatment of uveal melanoma comprising a mouse double minute 2 inhibitor (MDM2i) selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.

34. The compound according to claim 33, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof.

35. The compound according to claim 33, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.

36. A pharmaceutical combination comprising:
(i) a MDM2i, selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof,
and,
(ii) at least one protein kinase C pathway inhibitor (PKCi) selected from 3-(1.H.-indol-3-yl)-4-[2-(4-methyl-piperazin-1-yl)-quinazolin-4-yl]-pyrrole-2,5-dione, or a pharmaceutically acceptable salt thereof or 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof, 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof, and 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.

37. The pharmaceutical combination according to claim 36 for simultaneous, separate or sequential use.

38. The pharmaceutical combination according to 36 further comprising at least one pharmaceutically acceptable carrier.

39. The pharmaceutical combination according claim 36 in the form of a fixed combination.

40. The pharmaceutical combination according to claim 36 in the form of a kit of parts for combined administration, wherein the MDM2i and the PKCi are administered independently at the same time or separately within time intervals.

41. The pharmaceutical combination according to claim 36 wherein the MDM2i and PKCi are in a quantity which is jointly therapeutically effective for the treatment of uveal melanoma.

42. The pharmaceutical combination according claim 36 in the form of a combination product.

43. The pharmaceutical combination according to claim 36 for the treatment of uveal melanoma.

44. The pharmaceutical combination according to claim 43, wherein the uveal melanoma is metastatic uveal melanoma.

45. The pharmaceutical combination according to claim 43, wherein the uveal melanoma comprises metastasis of uveal melanoma.

46. The pharmaceutical combination according to claim 43, wherein the uveal melanoma comprises functional p53 or wild-type TP53.

47. The pharmaceutical combination according to claim 43, wherein the uveal melanoma is characterized by mutation of guanine nucleotide-binding protein G(q) subunit alpha (GNAQ) gene or guanine nucleotide-binding protein G(q) subunit 11 (GNA11) gene.

48. The pharmaceutical combination according to claim 44, wherein the metastatic uveal melanoma is characterized by mutation of guanine nucleotide-binding protein G(q) subunit alpha (GNAQ) gene or guanine nucleotide-binding protein G(q) subunit 11 (GNA11) gene.

49. The pharmaceutical combination according to claim 36, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof.

50. The pharmaceutical combination according to claim 36, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.

51. The pharmaceutical combination according to claim 36, wherein the PKCi is 3-(1.H.-indol-3-yl)-4-[2-(4-methyl-piperazin-1-yl)-quinazolin-4-yl]-pyrrole-2,5-dione, or a pharmaceutically acceptable salt thereof.

52. The pharmaceutical combination according to claim 36, wherein the PKCi is 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof.

53. The pharmaceutical combination according to claim 36, wherein the PKCi is 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof.

54. The pharmaceutical combination according to claim 36, wherein the PKCi is 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.

55. A method of treating a patient suffering from uveal melanoma or metastatic uveal melanoma comprising administering to the patient either simultaneously, separately or sequentially a therapeutically effective amount of
(i) a MDM2i, selected from (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, and (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof,
and,
(ii) at least one protein kinase C pathway inhibitor (PKCi) selected from 3-(1.H.-indol-3-yl)-4-[2-(4-methyl-piperazin-1-yl)-quinazolin-4-yl]-pyrrole-2,5-dione, or a pharmaceutically acceptable salt thereof or 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof, 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof, and 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.

56. The method of treating a patient according to claim 55, wherein the MDM2i is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof.

57. The method of treating a patient according to claim 55, wherein the MDM2i is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or a pharmaceutically acceptable salt thereof.

58. The method of treating a patient according to claim 55, wherein the PKCi is 3-(1.H.-indol-3-yl)-4-[2-(4-methyl-piperazin-1-yl)-quinazolin-4-yl]-pyrrole-2,5-dione, or a pharmaceutically acceptable salt thereof.

59. The method of treating a patient according to claim 55, wherein the PKCi is 3-amino-N-(3-(4-amino-4-methylpiperidin-1-yl)pyridin-2-yl)-6-(3-(trifluoromethyl)pyridin-2-yl)pyrazine-2-carboxamide, or a pharmaceutically acceptable salt thereof.

60. The method of treating a patient according to claim 55, wherein the PKCi is 3-amino-N-(3-(4-aminopiperidin-1-yl)pyridin-2-yl)-6-(3-(trifluoromethoxy)pyridin-2-yl)pyrazine-2-carboxamide, or a pharmaceutically acceptable salt thereof.

61. The method of treating a patient according to claim 55, wherein the PKCi is 3-amino-N-(3-(4-amino-4-methylpiperidin-1-yl)pyridin-2-yl)-6-(3-(trifluoromethoxy)pyridin-2-yl)pyrazine-2-carboxamide, or a pharmaceutically acceptable salt thereof.
